(19) United States
(12) Patent Application Publication
(10) Pub. No.: US 2026/0171241 A1
(43) Pub. Date: Jun. 18, 2026

(54) DIAGNOSIS SUPPORT DEVICE, DIAGNOSIS SUPPORT METHOD, AND PROGRAM

(71) Applicant: Sony Group Corporation, Tokyo (JP)

(72) Inventor: Satoshi OZAKI, Kanagawa (JP)

(73) Assignee: Sony Group Corporation, Tokyo (JP)

(21) Appl. No.: 18/853,420

(22) PCT Filed: Apr. 20, 2023
(86) PCT No.: PCT/JP2023/015775

§ 371 Date: Oct. 2, 2024

(30) Foreign Application Priority Data
Apr. 26, 2022 (JP) 2022-072197

(51) Int. Cl. G16H 50/20

(52) U.S. Cl. CPC G16H 50/20

(57) ABSTRACT

A diagnosis support device includes: a state determination unit that determines a state of a target of diagnosis; and a treatment necessity determination unit that determines whether diagnostic treatment is necessary on the basis of the state of the target of diagnosis.

### TECHNICAL FIELD

The present technique relates to a diagnosis support device, a diagnosis support method, and a program.

### BACKGROUND ART

Conventionally, techniques have been proposed to diagnose patients using machine learning or the like (PTL 1).

A technique described in PTL 1 is configured to categorize image data, which can be acquired from clinical diagnosis equipment by using a mechanical learning technique, on the basis of a method conforming to a diagnosis of an ophthalmologist, and derive the disease of a patient from features in the image data.

### CITATION LIST

### Patent Literature

PTL 1: JP 2020-36835A

### SUMMARY

### Technical Problem

The technique of PTL 1 is configured to derive the disease of a patient, that is, to conduct a diagnosis. In some diagnoses, some kind of diagnostic treatment may need to be performed on the patient to conduct a more accurate diagnosis.

Unfortunately, the conventional art of PTL 1 cannot determine the necessity for such diagnostic treatment, which has been left unsolved.

The present technique has been devised in view of such a problem. An object of the present technique is to provide a diagnosis support device, a diagnosis support method, and a program that can support diagnosis by determining whether diagnostic treatment is necessary.

### Solution to Problem

In order to solve the problem, a first technique is a diagnosis support device including: a state determination unit that determines a state of a target of diagnosis; and a treatment necessity determination unit that determines whether diagnostic treatment is necessary on the basis of the state of the target of diagnosis.

Moreover, a second technique is a diagnosis support method including:
determining a state of a target of diagnosis; and determining whether diagnostic treatment is necessary on the basis of the state of the target of diagnosis.

Furthermore, a third technique is a program that causes a computer to perform a diagnosis support method including: determining a state of a target of diagnosis; and determining whether diagnostic treatment is necessary on the basis of the state of the target of diagnosis.

### BRIEF DESCRIPTION OF DRAWINGS

FIG. 1 is a block diagram illustrating the configuration of an electronic device 10.

FIG. 2A illustrates a state of photographing using a camera 17 of the electronic device 10, and FIG. 2B illustrates an example of an image obtained by photographing.

FIG. 3 is a block diagram illustrating the configuration of a diagnosis support device 100 according to a first embodiment.

FIG. 4 is a flowchart showing the processing of the diagnosis support device 100 according to the first embodiment.

FIG. 5 is an explanatory drawing showing the reliability calculation of a diagnosis result.

FIG. 6 shows an instillation instruction displayed on a display unit 15.

FIG. 7 shows a consultation instruction displayed on the display unit 15.

FIG. 8 is a block diagram illustrating the configuration of an information processing system 1000 according to a second embodiment.

FIG. 9 is a block diagram illustrating the configuration of a relay server 20.

FIG. 10 is a block diagram illustrating the configuration of a diagnosis support device 200 according to the second embodiment.

FIG. 11 is a flowchart showing the processing of the diagnosis support device 200 according to the second embodiment.

FIG. 12 shows a mydriatic-agent instillation unnecessity instruction displayed on the display unit 15.

FIG. 13 is a block diagram illustrating the configuration of an information processing system 2000 according to a third embodiment.

FIG. 14 is a block diagram illustrating the configuration of an administrative server 30.

FIG. 15 is a flowchart showing the processing of the administrative server 30.

### DESCRIPTION OF EMBODIMENTS

Embodiments of the present technique will be described below with reference to the drawings. Descriptions will be made in the following order:
<1. First embodiment>
[1-1. Configuration of electronic device 10]
[1-2. Configuration of diagnosis support device 100]
[1-3. Processing in diagnosis support device 100]
<2. Second embodiment>
[2-1. Configuration of information processing system 1000]
[2-2. Configuration of diagnosis support device 200]
[2-3. Processing in diagnosis support device 200]
<3. Third embodiment>
[3-1. Configuration of information processing system 2000]
[3-2. Processing in diagnosis support device 200 and processing in administrative server 30]
<4. Modification example>

### 1. First Embodiment

### [1-1. Configuration of Electronic Device 10]

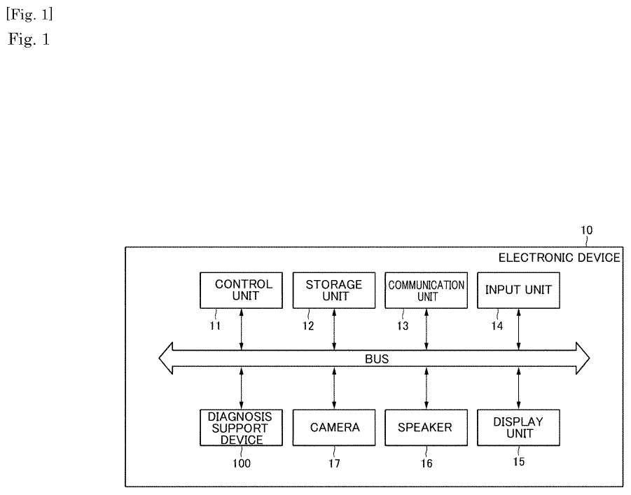

Referring to FIG. 1, the configuration of an electronic device 10, in which a diagnosis support device 100 according to the present technique operates, will be described below. The electronic device 10 includes a control unit 11, a storage unit 12, a communication unit 13, an input unit 14, a display unit 15, a speaker 16, and a camera 17.

The control unit 11 includes a CPU (Central Processing Unit), a RAM (Random Access Memory), and a ROM (Read Only Memory). The CPU controls the overall electronic device 10 and each unit thereof by performing various types of processing according to programs stored in the ROM and issuing commands.

The storage unit 12 stores various types of information input from the input unit 14, diagnosis results, and programs or the like. The storage unit 12 is, for example, a large-capacity storage medium such as a hard disk or a flash memory.

The communication unit 13 is a communication interface between the electronic device 10 and external devices and the Internet. The communication unit 13 may include a wire or radio communication interface. More specifically, the wire or radio communication interface may include cellular communications, Wi-Fi, Bluetooth (registered trademark), NFC (Near Field Communication), Ethernet (registered trademark), HDMI (registered trademark), (High-Definition Multimedia Interface), and USB (Universal Serial Bus).

The input unit 14 allows a user to input instructions or the like to the electronic device 10. In response to a user input to the input unit 14, a control signal corresponding to the input is generated and is supplied to the control unit 11.

The control unit 11 then performs various types of processing in response to the control signal. The input unit 14 may be a touch panel or a touch screen integrated with the display unit 15, as well as a physical button.

The display unit 15 is, for example, a liquid crystal display or an organic EL (Electroluminescence) display that displays a through image during photographing using the camera 17, an instillation instruction output from the diagnosis support device 100, a consultation instruction, and the operation screen of the electronic device 10.

The speaker 16 is configured to output sound. In the present technique, an instillation instruction and a consultation instruction may be output as sound, which will be specifically described later. The electronic device 10 may be configured without the speaker 16.

The camera 17 is a camera that includes an imaging element and a signal processing circuit and is capable of photographing still images and moving images. Additionally, the camera 17 may be configured to be connected as a separate device of the electronic device 10 to the electronic device 10 via wired or wireless connection.

The electronic device 10 is configured thus. For example, the electronic device 10 is a smartphone, a tablet, a personal computer, or a wearable device.

When a program necessary for processing according to the present technique is provided, the program may be installed in the electronic device 10 in advance or may be installed by a user after being downloaded or distributed in a storage medium or the like.

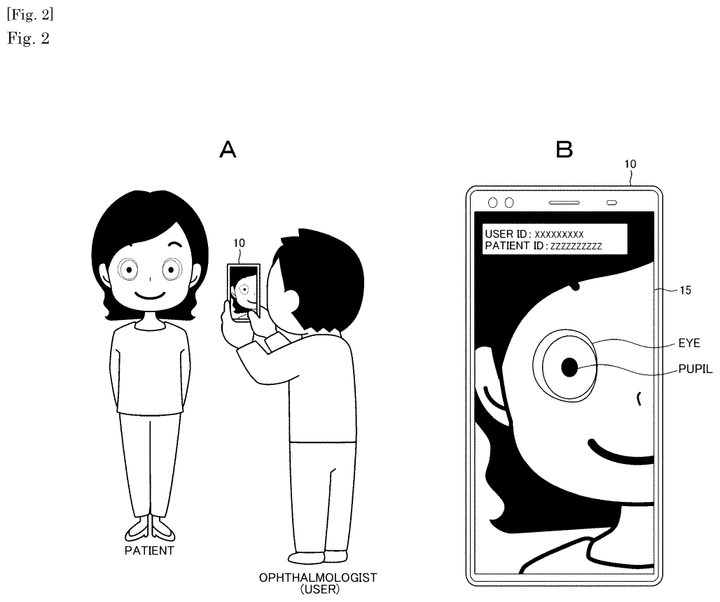

In a first embodiment, an ophthalmologist as a user of the electronic device 10 photographs a patient's eye with the camera 17 as illustrated in FIG. 2A and then inputs an image to the diagnosis support device 100, the image including the patient's eye as illustrated in FIG. 2B. In the following description of the first embodiment, an ophthalmologist using the electronic device 10 may be referred to as a user.

### [1-2. Configuration of Diagnosis Support Device 100]

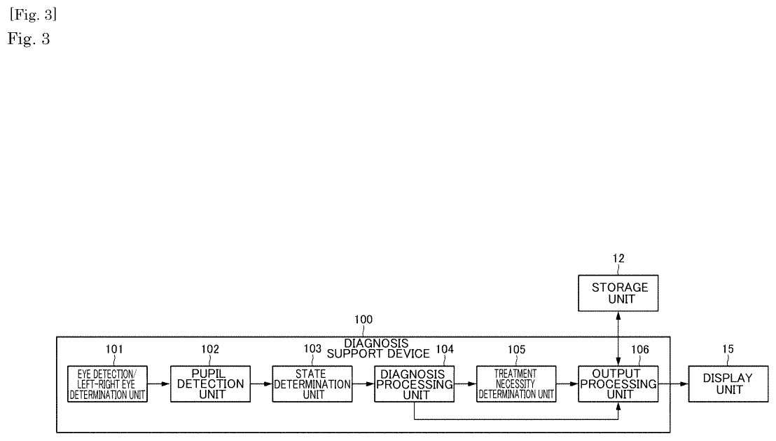

Referring to FIG. 3, the configuration of the diagnosis support device 100 will be described below. The diagnosis support device 100 includes an eye detection/left-right eye determination unit 101, a pupil detection unit 102, a state determination unit 103, a diagnosis processing unit 104, a treatment necessity determination unit 105, and an output processing unit 106.

The diagnosis support device 100 determines the state of the pupil of a user's eye that is detected as a target of diagnosis from an input image captured by the camera 17 and diagnoses an eye disease according to the state of the pupil. In first to third embodiments, the eye disease is assumed to be a cataract.

Furthermore, the diagnosis support device 100 determines whether the instillation of a mydriatic agent is necessary as diagnostic treatment for diagnosing a cataract. When necessary, an ophthalmologist is instructed to instill the mydriatic agent.

The mydriatic agent is an agent for temporarily dilating a pupil to diagnose an eye disease in detail. This because an illuminated pupil constricts and becomes difficult to diagnose. In the present technique, a state in which a pupil is dilated is referred to as a mydriatic state while a state in which a pupil is not dilated is referred to as a non-mydriatic state. For example, a pupil having a diameter of 5 mm or larger can be assumed to be placed in a mydriatic state while a pupil having a diameter smaller than 5 mm can be assumed to be placed in a non-mydriatic state. This criterion is merely exemplary, and the present technique is not limited to a specific criterion for mydriasis and non-mydriasis. Although diagnosis of cataract requires the instillation of a mydriatic agent, dilated pupils may cause low visibility for several days or produce side effects. Thus, the instillation of a mydriatic agent is preferably minimized. In the instillation of a mydriatic agent, it is preferable to correctly use the mydriatic agent for an eye to be diagnosed and record the correct use. Conventionally, ophthalmologists and nurses have no alternative other than to carefully conduct instillation.

The eye detection/left-right eye determination unit 101 detects an eye to be diagnosed from an image that is captured by the camera 17 and is input to the diagnosis support device 100. Moreover, whether the detected eye is a right eye or a left eye is determined. The eye detection/left right eye determination unit 101 outputs the detection result to the pupil detection unit 102. The eye detection/left right eye determination unit 101 may be configured as separate processing blocks serving as an eye detection unit and a left right eye determination unit.

The pupil detection unit 102 detects a pupil in the eye detected from the image. The pupil detection unit 102 outputs the detection result to the state determination unit 103. The eye detection/left right eye determination unit 101 and the pupil detection unit 102 are equivalent to a diagnosis target detection unit in the claims.

The state determination unit 103 determines whether the detected pupil of the eye to be diagnosed is placed in a mydriatic state or in a non-mydriatic state.

The state determination unit 103 outputs the determination result to the diagnosis processing unit 104.

The diagnosis processing unit 104 diagnoses an eye disease on the basis of the pupil of the eye detected from the image. The diagnosis processing unit 104 has a mydriasis diagnostic model used when a pupil is placed in a mydriatic state and a non-mydriasis diagnostic model used when a pupil is placed in a non-mydriatic state. The diagnostic models to be used are switched on the basis of the determination result of the state determination unit 103. The diagnosis processing unit 104 outputs the diagnosis result to the treatment necessity determination unit 105 and the output processing unit 106.

The eye detection/left right eye determination unit 101, the pupil detection unit 102, the state determination unit 103, and the diagnosis processing unit 104 can be implemented using a learned model having undergone machine learning using a correct label and an image. Alternatively, these units can also be implemented machine learning algorithm.

The treatment necessity determination unit 105 determines whether the instillation of a mydriatic agent is necessary as diagnostic treatment on the basis of the state of the eye to be diagnosed and the diagnosis result. The state of the eye refers to a mydriatic state or a non-mydriatic state. The treatment necessity determination unit 105 further determines whether a patient needs to consult an ophthalmologist on the basis of the diagnosis result. The treatment necessity determination unit 105 outputs the determination result to the output processing unit 106.

The output processing unit 106 associates the diagnosis result obtained by the diagnosis processing unit 104 with information including a user ID, a patient ID, eye left/right information, mydriatic state information, and diagnosis result reliability, and outputs the diagnosis result to the storage unit 12. The diagnosis result output to the storage unit 12 is stored as a diagnosis record.

Diagnosis records may be stored collectively in a server or the like on a network, in addition to the storage unit 12. It is assumed that the user ID and the patient ID are input in advance by, for example, an ophthalmologist as a user through the input unit 14. The user ID and the patient ID may be acquired from a hospital database or the like.

When the treatment necessity determination unit 105 determines that the instillation of a mydriatic agent is necessary as diagnostic treatment, the output processing unit 106 outputs an instillation instruction to the display unit 15.

Furthermore, when the treatment necessity determination unit 105 determines that a patient needs to consult an ophthalmologist, the output processing unit 106 outputs a consultation instruction, which instructs the patient to consult the ophthalmologist, to the display unit 15. The instillation instruction and the consultation instruction are displayed on the display unit 15, so that the instructions are presented to the ophthalmologist and the patient. The output processing unit 106 may output the instillation instruction and the consultation instruction to the speaker 16. In this case, the instillation instruction and the consultation instruction are output as sound from the speaker 16, so that the instructions are presented to the ophthalmologist and the patient.

The diagnosis support device 100 is configured thus. In the present embodiment, the diagnosis support device 100 operates in the electronic device 10. The electronic device 10 may have the function of the diagnosis support device 100 in advance, or the electronic device 10 having the function of a computer may execute a program to implement the diagnosis support device 100 and a diagnosis support method. Alternatively, the control unit 11 may be allowed to act as the diagnosis support device 100 by executing a program. The program may be installed as an eye-disease diagnosis application in the electronic device 10 in advance or may be installed by a user or others after being downloaded or distributed in a storage medium or the like. Alternatively, the diagnosis support device 100 may be configured as a single device.

### [1-3. Processing by Diagnosis Support Device 100]

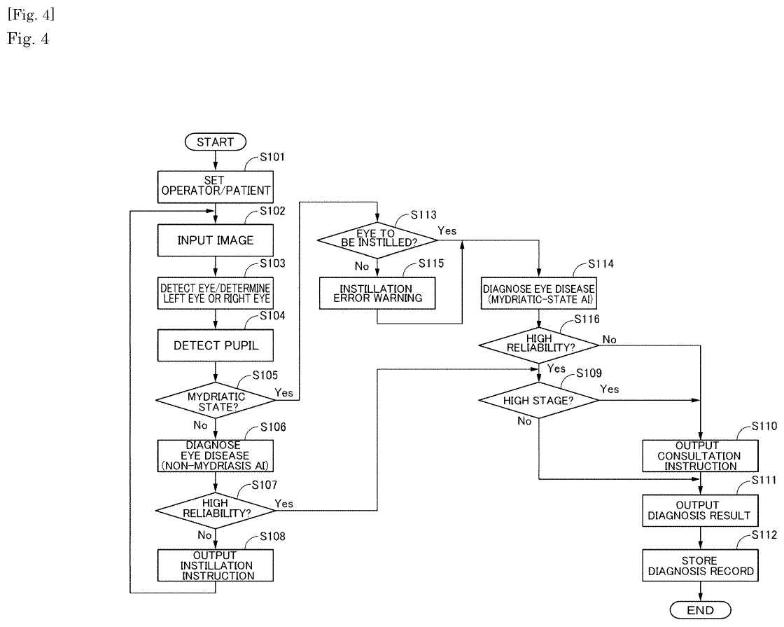

Referring to FIG. 4, processing by the diagnosis support device 100 will be described below.

First, in step S101, a user and a patient are set on the basis of the user ID and the patient ID that are input through the input unit 14. As described above, the user is an ophthalmologist in the first embodiment.

Subsequently, in step S102, an image captured by the camera 17 is input to the diagnosis support device 100. As illustrated in FIG. 2, photographing needs to be performed while the ophthalmologist as a user points the camera 17 of the electronic device 10 at the face of a patient to place a patient's eye in the angle of view.

For example, the image is captured as illustrated in FIG. 2B. As illustrated in FIG. 2B, the image is preferably displayed on the display unit 15 while being associated with the user ID and the patient ID that can identify the user and the patient. Furthermore, the patient may be identified using face recognition technology or iris authentication technology, and the identification result associated with the image may be displayed.

In step S103, the eye detection/left right eye determination unit 101 detects an eye and determines whether the eye is a left eye or a right eye on the basis of the image.

In step S104, the pupil detection unit 102 detects a pupil on the basis of the eye in the image, the eye being detected by the eye detection/left right eye determination unit 101.

In step S105, the state determination unit 103 determines whether the eye in the image is placed in a mydriatic state or a non mydriatic state. When the determination result is a non-mydriatic state, the processing advances to step S106 (No at step S105).

In step S106, the diagnosis processing unit 104 diagnoses an eye disease on the basis of the image of the eye. When the processing advances to step S106, the eye is placed in a non-mydriatic state. Thus, the diagnosis processing unit 104 diagnoses an eye disease by using the non-mydriasis model.

Subsequently, in step S107, the treatment necessity determination unit 105 determines whether the diagnosis result obtained by the diagnosis processing unit 104 has high reliability. When the treatment necessity determination unit 105 determines that the diagnosis result has low reliability, the processing advances to step S108 (No at step S107).

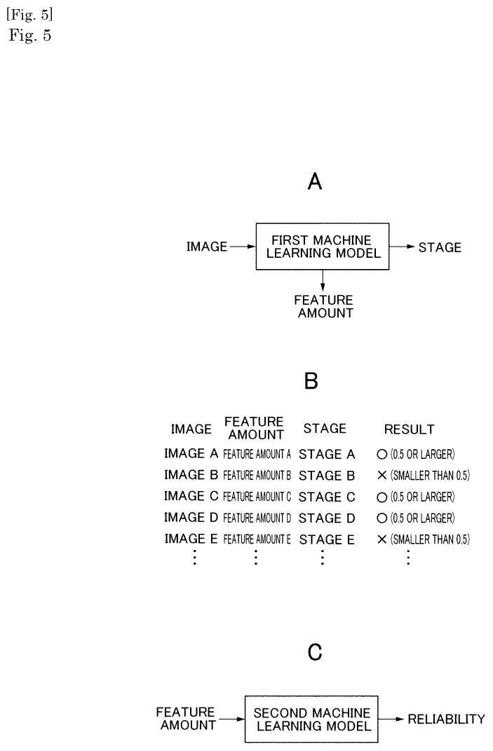

The reliability of the diagnosis result will be described below. First, as shown in FIG. 5A, the image of the eye is input to a first machine learning model for diagnosing an eye disease. A feature amount of feature extraction in the image of the eye is output from the first machine learning model. Moreover, the first machine learning model infers the stage of an eye disease, the stage corresponding to the feature amount extracted from the image of the eye, and then the stage is also output. The feature amount refers to information for outputting the stage of a disease.

The feature amount and the stage that are output from the first machine learning model are associated with each other. As shown in FIG. 5B, pairs of feature amounts and stages can be obtained as many as images input to the first machine learning model. The stage may correspond to or differ from an actual symptom. A matching result on whether the stage matches an actual symptom is also associated with the pair of the feature amount and the stage. The matching result is denoted as ○ or x. For example, the matching result is output as a numeric value of 0 to 1. When the numeric value is 0.5 or larger, the result is denoted as ○. When the numeric value is smaller than 0.5, the result is denoted as x. High reliability is, for example, the reliability of 0.5 or higher. The value is merely exemplary and the present technique is not limited to this example.

Subsequently, as shown in FIG. 5C, the feature amount output by the first machine learning model is input to a second machine learning model. The second machine learning model is a model for estimating whether the stage corresponding to the feature amount output by the first machine learning model matches an actual symptom. Hence, the input of multiple feature amounts allows the second machine learning model to learn “the stage matches an actual symptom at this feature amount” and “the stage does not match an actual symptom at this feature amount”. By using the second machine learning model that has been learned thus, reliability indicating whether the stage matches an actual symptom can be output from a feature amount extracted from an additionally input image of an eye. Moreover, the matching result may be a difference in value between a diagnostic stage and an actual stage. For example, the matching result is learning of 0 or 1 or reliability is inferred as a value of 0.0 to 1.0, indicating that the reliability increases as the value approaches 1.0. When a difference is used, the value increases as the reliability decreases. The value may reach 2.0 or 3.0. In this case, for example, reliability is obtained at 0.5 or less.

Returning to the flowchart of FIG. 4, in step S108, the output processing unit 106 outputs an instillation instruction, which instructs an ophthalmologist to instill a mydriatic agent to a patient, to the display unit 15. The instillation instruction includes information about an eye (right eye or left eye) as a target of instillation.

The instillation instruction is output in step S108 because a diagnosis is conducted in a non-mydriatic state and then in the case of a less reliable diagnosis result, another diagnosis is to be conducted after the mydriatic agent is instilled to place an eye into a mydriatic state. As described above, a diagnosis is conducted on the basis of an eye in a non mydriatic state, and the instillation of the mydriatic agent is instructed only when the diagnosis result has low reliability. This can prevent unnecessary use and wrong use of the mydriatic agent.

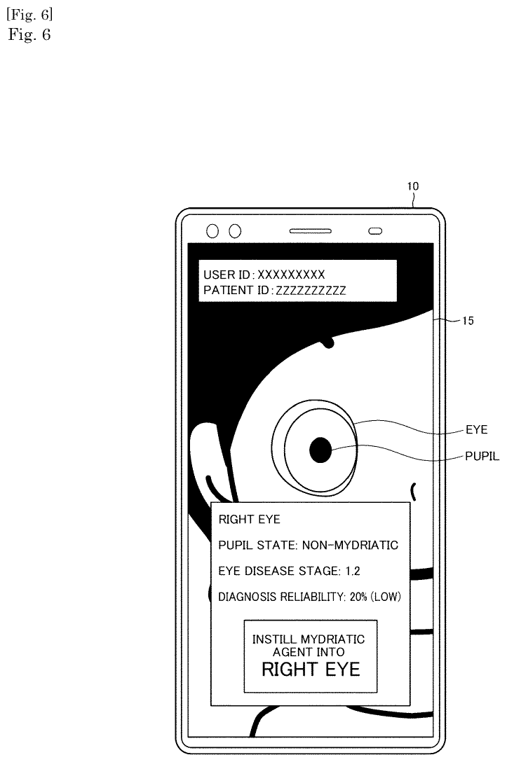

For example, the instillation instruction is displayed on the display unit 15 as illustrated in FIG. 6. According to the instruction, the ophthalmologist instills the mydriatic agent to the patient. A visible range in a pupil is physically larger in a mydriatic state, and thus information supporting diagnosis increases to facilitate diagnosis. When the instillation instruction is displayed, an eye as a target of instillation is preferably indicated with a reminder in order to prevent misuse of a mydriatic agent. Methods for indicating an eye as a target of instillation include thickening letters indicating an eye, upsizing the letters, and coloring the letters differently from other characters.

After the output processing unit 106 outputs the instillation instruction to the display unit 15 in step S108, the ophthalmologist as a user photographs the eye again using the camera 17, the eye being instilled with the mydriatic agent. In step S102, the image is input to the diagnosis support device 100. Hence, the display unit 15 may display an instruction to capture another image of the eye.

When the treatment necessity determination unit 105 determines that the diagnosis result has high reliability in step S107, the processing advances to step S109 (Yes at step S107).

Subsequently, in step S109, the treatment necessity determination unit 105 determines whether an eye disease has advanced to a high stage with reference to the diagnosis result. If the eye disease has advanced to a high stage, the processing advances to step S110 (Yes at step S109).

The stage is an index generally used in diagnosis of cataract and is represented as a numeric value of 0 to 5. However, the stage may be expressed by another index. The stage of cataract may be expressed by indexes varying among countries or regions. In the first to third embodiments, a stage at 3 or higher is assumed to be advanced, whereas a stage lower than 3 is assumed to be less advanced. This criterion is merely exemplary, and the present technique is not limited to this criterion.

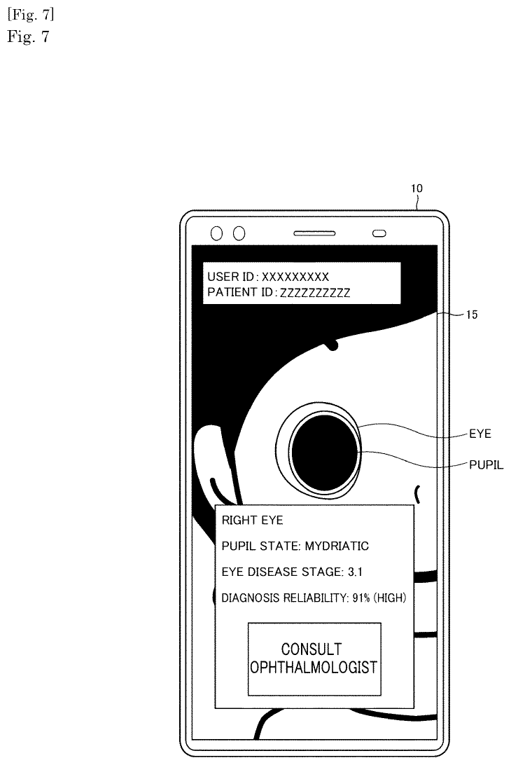

Subsequently, in step S110, the output processing unit 106 outputs a consultation instruction, which instructs the patient to consult the ophthalmologist, to the display unit 15. For example, the consultation instruction is displayed on the display unit 15 as illustrated in FIG. 7.

The consultation instruction is provided thus because the patient should consult the ophthalmologist when the diagnosis result obtained by the diagnosis processing unit 104 has high reliability and the stage of an eye disease is advanced.

Subsequently, in step S111, the output processing unit 106 outputs the diagnosis result to the display unit 15, so that the diagnosis result is displayed on the display unit 15 as illustrated in, for example, FIGS. 6 and 7. In the examples of FIGS. 6 and 7, a user ID, a patient ID, an eye image, eye left/right information, mydriatic state information, the stage of an eye disease, and diagnosis reliability are displayed. The output processing unit 106 may output the diagnosis result to the speaker 16. In this case, the diagnosis result is output as sound from the speaker 16.

Subsequently, in step S112, the output processing unit 106 associates the diagnosis result with the user ID, the patient ID, the eye left/right information, the mydriatic state information, and the reliability and outputs the diagnosis result to the storage unit 12, so that the diagnosis result is stored as a diagnosis record.

When the treatment necessity determination unit 105 determines that the diagnosis result is a less advanced stage in step S109, the processing advances to Returning to step S105, when the state determination unit 103 determines that the eye in the image is placed in a mydriatic state in step S105, the processing advances to step S113 (Yes at step S105).

Subsequently, in step S113, it is confirmed whether the eye in the image is to be instilled with the mydriatic agent. Whether the eye is to be instilled can be confirmed by referring to the detection result of the eye detection/left-right eye determination unit 101 and information about eyes to be instilled in the instillation instruction.

When the eye in the image is a target of the instillation instruction, the processing advances to step S114 (Yes at step S113).

When the eye in the image is not a target of the instillation instruction in step S113, the processing advances to step S115 (No at step S113). When the eye is placed in a mydriatic state, whether the eye is a target of the instillation instruction in step S113. When the eye is not a target of the instillation instruction, a warning is issued in step S115. The warning can display a predetermined message or icon on the display unit 15 or output a predetermined voice message from the speaker 16. After the warning is displayed, the processing advances to step S114.

Subsequently, in step S114, the diagnosis processing unit 104 diagnoses an eye disease on the basis of the image of the eye. When the processing advances to step S114, the eye is placed in a mydriatic state. Thus, the diagnosis processing unit 104 diagnoses an eye disease by using the mydriasis model.

Subsequently, in step S116, the treatment necessity determination unit 105 determines whether the diagnosis result has high reliability. If the diagnosis result has reliability, the processing advances to step S109 (Yes at step S116). Thereafter, the processing of steps S109 to S112 is performed as in the foregoing description.

When the treatment necessity determination unit 105 determines that the diagnosis result has low reliability in step S116, the processing advances to step S110 (No at step S116). In this case, the processing of steps S110 to S112 is performed as in the foregoing description.

When it is determined in step S105 again that the eye is placed in a non-mydriatic state after the instillation instruction is output in step S108, the display unit 15 may display a warning that the eye has not been placed into a mydriatic state after the instillation of the mydriatic agent.

The case of the processing of step S112 performed after the instillation instruction is output in step S108 is a case where the eye is changed from a non-mydriatic state to a mydriatic state by the instillation of the mydriatic agent. Thus, the process is stored as a diagnosis record, keeping a history of the use of the mydriatic agent and a change of a pupil into a mydriatic state by using the mydriatic agent.

The processing of the diagnosis support device 100 in the first embodiment is performed thus. According to the first embodiment, only when a patient's eye is placed in a non-mydriatic state and the diagnosis result has low reliability, an instruction to instill the mydriatic agent is provided. Thus, even when a patient's eye is placed in a non-mydriatic state, an instruction to instill the mydriatic agent is not provided for a diagnosis result having high reliability. This can avoid unnecessary use of the mydriatic agent. Moreover, it can be determined whether an eye to be diagnosed has been placed into a mydriatic state by the instillation of the mydriatic agent, and the presence or absence of the instillation of the mydriatic agent and the mydriatic state and others can be recorded with the diagnosis result. The diagnosis processing unit 104 diagnoses an eye disease, reducing diagnoses conducted by an ophthalmologist. Moreover, the diagnosis processing unit 104 can provide information for diagnosing an eye disease by an ophthalmologist. Furthermore, an eye to be instilled with the mydriatic agent can be indicated, providing a reminder to prevent erroneous instillation.

### 2. Second Embodiment

### [2-1. Configuration of Information Processing System 1000]

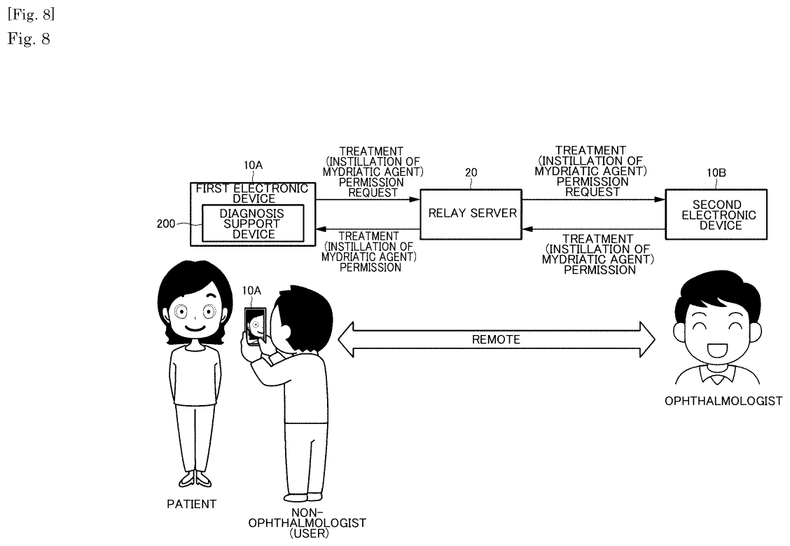

A second embodiment according to the present technique will be described below. As illustrated in FIG. 8, an information processing system 1000 according to the second embodiment includes a first electronic device 10A, a relay server 20, and a second electronic device 10B.

The second embodiment is configured such that a person other than an ophthalmologist (referred to as a non-ophthalmologist) photographs a patient's eye using the first electronic device 10A in a state in which the ophthalmologist is located at a remote site from a patient and does not face the patient, and a diagnosis support device 200 diagnoses an eye disease and determines the necessity for the instillation of a mydriatic agent. The non-ophthalmologist may be any person, for example, a doctor such as an internist other than an ophthalmologist, a nurse, a care worker, a patient's family member, or a patient as long as the patient can receive an instruction from an ophthalmologist.

In a specific case, for example, a non-ophthalmologist having visited a patient diagnoses the eye disease of the patient and determines the necessity for the instillation of a mydriatic agent by using the first electronic device 10A. In another case, for example, an ophthalmologist is remote from a hospital and a non-ophthalmologist at the hospital diagnoses the eye disease of a patient and determines the necessity for the instillation of a mydriatic agent by using the first electronic device 10A.

In such circumstances, a non-ophthalmologist facing a patient is not an ophthalmologist and thus cannot make a decision to instill a mydriatic agent to the patient. The instillation of a mydriatic agent by a non-ophthalmologist requires permission from an ophthalmologist. Thus, the second embodiment provides a mechanism that allows an ophthalmologist at a remote site from a patient to permit the instillation of a mydriatic agent.

The first electronic device 10A and the second electronic device 10B are configured like the electronic device 10 of the first embodiment, and thus the description thereof is omitted.

The first electronic device 10A has the function of the diagnosis support device 200 as in the first embodiment. The first electronic device 10A is used by a non-ophthalmologist and transmits instillation permission request information when the diagnosis support device 200 determines that the instillation of a mydriatic agent is necessary. The instillation permission request information is information for requesting permission of the instillation of the mydriatic agent. In the following description, the non-ophthalmologist using the first electronic device 10A may be referred to as a user.

The second electronic device 10B is used by an ophthalmologist and is configured to confirm the received instillation permission request information and transmit permission of the instillation of the mydriatic agent.

The second electronic device 10B is used by the ophthalmologist to confirm the instillation permission request information and transmit information about permission of the instillation of the mydriatic agent (instillation permission information). The second electronic device 10B may be provided without a camera function because photographing a patient with a camera is not necessary.

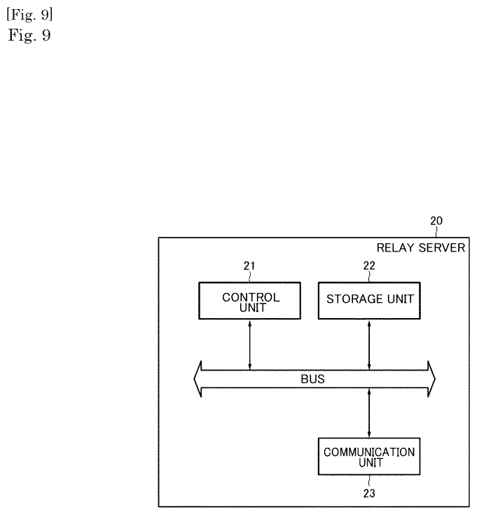

As illustrated in FIG. 9, the relay server 20 includes at least a control unit 21, a storage unit 22, and a communication unit 23. The control unit 21, the storage unit 22, and the communication unit 23 have the same functions as the electronic device 10 of the first embodiment.

The relay server 20 has an ordinary communication function and is configured to relay the transmission and reception of information between the first electronic device 10A and the second electronic device 10B via a network or the Internet. The relay server 20 may record information transmitted and received between the first electronic device 10A and the second electronic device 10B, by using the storage unit 22 provided in the relay server 20 or an external device connected via a network.

### [2-2. Configuration of Diagnosis Support Device 200]

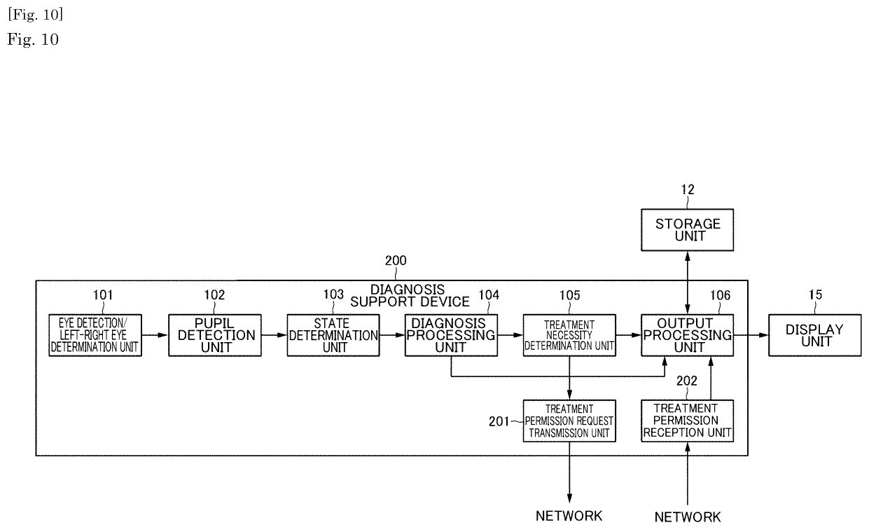

The configuration of the diagnosis support device 200 according to the second embodiment will be described below. As shown in FIG. 10, the diagnosis support device 200 includes an eye detection/left right eye determination unit 101, a pupil detection unit 102, a state determination unit 103, a diagnosis processing unit 104, a treatment necessity determination unit 105, an output processing unit 106, a treatment permission request transmission unit 201, and a treatment permission reception unit 202. The eye detection/left right eye determination unit 101, the pupil detection unit 102, the state determination unit 103, the diagnosis processing unit 104, the treatment necessity determination unit 105, and the output processing unit 106 are configured as in the first embodiment, and thus the description thereof is omitted.

When the treatment necessity determination unit 105 determines that the instillation of the mydriatic agent is necessary as diagnostic treatment, the treatment permission request transmission unit 201 transmits instillation permission request information to the relay server 20 via a network. The instillation permission request information is transmitted to the second electronic device 10B by the relay server 20. The instillation permission request information includes a user ID, a patient ID, an ophthalmologist ID, eye left/right information, a mydriatic state determination result, a diagnosis result, diagnosis reliability, and an eye image. However, the instillation permission request information may include other kinds of information.

The treatment permission reception unit 202 receives instillation permission information transmitted from the second electronic device 10B via the network and the relay server 20 and supplies the information to the output processing unit 106.

The instillation permission information includes a user ID, a patient ID, an ophthalmologist ID, eye left/right information, and the validity period of treatment permission. However, the instillation permission information may include other kinds of information.

The diagnosis support device 200 is configured thus. As in the first embodiment, the electronic device 10 may have the function of the diagnosis support device 200 in advance, or the electronic device 10 having the function of a computer may execute a program to implement the diagnosis support device 200 and a diagnosis support method.

### [2-3. Processing by Diagnosis Support Device 200]

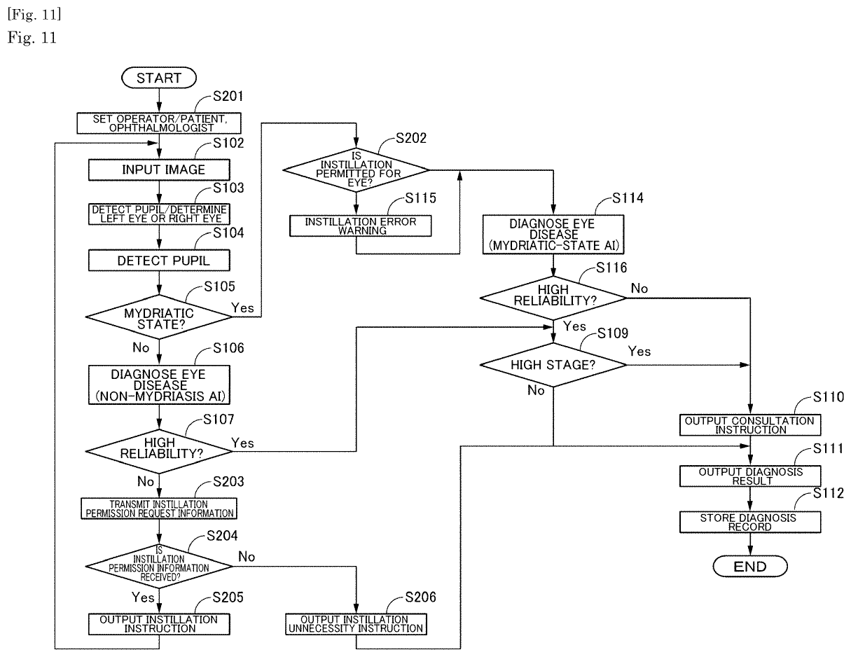

Referring to FIG. 11, processing by the diagnosis support device 200 according to the second embodiment will be described below. First, in step S201, a user (non-ophthalmologist), a patient, and an ophthalmologist are set on the basis of the user ID, the patient ID, and the ophthalmologist ID that are input through an input unit 14.

Steps S102 to S116 are similar to those of the first embodiment, and thus the description thereof is omitted.

In the second embodiment, whether instillation has been permitted for an eye is determined in step S202 instead of step S113 in the first embodiment.

When the treatment necessity determination unit 105 determines that the diagnosis result has low reliability in step S107, the processing advances to step S203 (No at step S107).

Subsequently, in step S203, the treatment permission request transmission unit 201 transmits the instillation permission request information. The instillation permission request information is transmitted to the second electronic device 10B via the relay server 20. As described above, when the diagnosis result has low reliability, that is, the instillation of the mydriatic agent is necessary as diagnostic treatment for detailed diagnosis, the instillation permission request information is transmitted to the second electronic device 10B used by an ophthalmologist. This is because a user in front of a patient is a non-ophthalmologist who is not an ophthalmologist and the non-ophthalmologist cannot make a decision to instill the mydriatic agent to the patient.

When confirming the contents of the instillation permission request information through the second electronic device 10B to permit the instillation of the mydriatic agent, the ophthalmologist operates the second electronic device 10B to transmit instillation permission information.

Subsequently, in step S204, when the treatment permission reception unit 202 receives the instillation permission information transmitted from the second electronic device 10B, the processing advances to step S205 (Yes at step S204).

Thereafter, in step S204, the output processing unit 106 outputs an instillation instruction, which instructs the non-ophthalmologist to instill the mydriatic agent to the patient, to the display unit 15. For example, the instillation instruction is displayed on the display unit 15 as illustrated in FIG. 6. According to the instruction, the non-ophthalmologist instills the mydriatic agent to the patient.

In step S203, when the treatment permission reception unit 202 does not receive the instillation permission information transmitted from the second electronic device 10B, the processing advances to step S206 (No at step S204). The case where the treatment permission reception unit 202 does not receive the instillation permission information is, for example, the case where the instillation permission information has not been received for a predetermined period after the transmission of the instillation permission request information. In addition to the case of the instillation permission information unreceived for a predetermined period, the same processing as in the case of the unreceived instillation permission information may be performed in the case where information about an instillation permission request rejected by the second electronic device 10B is transmitted and received by the first electronic device 10A.

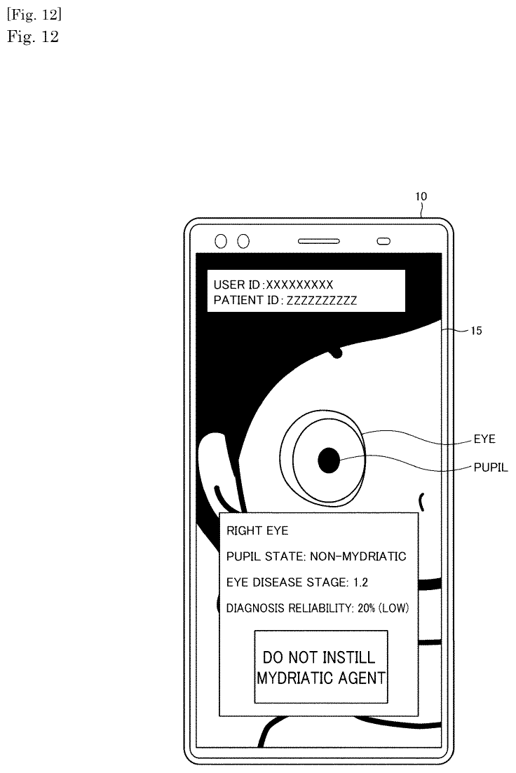

Subsequently, in step S206, the output processing unit 106 outputs an instillation unnecessity instruction, which instructs the non-ophthalmologist not to instill the mydriatic agent, to the display unit 15. The instillation unnecessity instruction is output to the display unit 15 by the output unit and is displayed on, for example, the display unit 15 as illustrated in FIG. 12. According to the instruction, the non-ophthalmologist does not instill the mydriatic agent to the patient. In the display example of FIG. 12, characters indicating a reason such as a time-out or unavailability of instillation permission may be displayed.

After step S206, step S111 and step S112 are performed as in the first embodiment.

The processing of the diagnosis support device 200 in the second embodiment is performed thus. According to the second embodiment, a proper instillation instruction of the mydriatic agent can be provided even when the ophthalmologist does not face the patient in the same space. This can eliminate the need for the ophthalmologist to face the patient to instill the mydriatic agent, thereby reducing spatial and time constraints to the ophthalmologist. Moreover, the patient can be diagnosed with high reliability even when the ophthalmologist is not present in the same space as the patient.

### 3. Third Embodiment

### [3-1. Configuration of Information Processing System 2000]

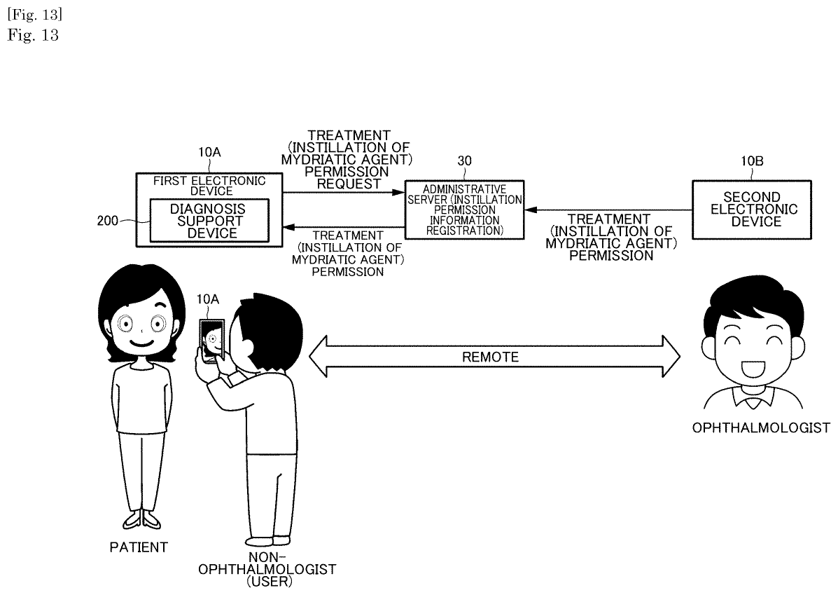

A third embodiment of the present technique will be described below. As illustrated in FIG. 13, an information processing system 2000 according to the third embodiment includes a first electronic device 10A, an administrative server 30, and a second electronic device 10B.

As in the second embodiment, the third embodiment is configured such that a non-ophthalmologist photographs a patient using the first electronic device 10A in a state in which an ophthalmologist is located at a remote site from a patient and does not face the patient, and a diagnosis support device 200 diagnoses an eye disease and determines the necessity for the instillation of a mydriatic agent. The specific case of the third embodiment is similar to that of the second embodiment.

In such circumstances, the non-ophthalmologist facing the patient cannot make a decision to instill the mydriatic agent to the patient, and the instillation of the mydriatic agent to the patient by the non ophthalmologist requires permission from the ophthalmologist. Thus, the third embodiment provides a mechanism in which an ophthalmologist preregisters instillation permission information in the administrative server 30, allowing the administrative server 30 to determine whether the instillation of a mydriatic agent is necessary and transmit the instillation permission information even when the ophthalmologist does not determine the necessity in real time.

The first electronic device 10A and the second electronic device 10B are configured like the electronic device 10 of the first embodiment, and thus the description thereof is omitted. Moreover, the diagnosis support device 200 is configured as in the second embodiment, and thus the description thereof is omitted.

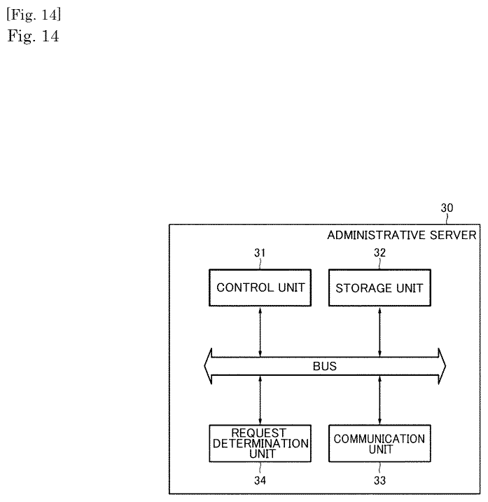

As illustrated in FIG. 14, the administrative server 30 includes at least a control unit 31, a storage unit 32, a communication unit 33, and a request determination unit 34. The control unit 31, the storage unit 32, and the communication unit 33 embodiment.

The ophthalmologist using the second electronic device 10B transmits instillation permission information to the administrative server 30 and preregisters the information therein. The instillation permission information transmitted from the second electronic device 10B is stored in advance in the storage unit 32 of the administrative server 30. The instillation permission information is similar to that of the second embodiment.

The administrative server 30 has an ordinary communication function and is configured to relay the transmission and reception of information between the first electronic device 10A and the second electronic device 10B via a network or the Internet. Additionally, the administrative server 30 may record information transmitted and received between the first electronic device 10A and the second electronic device 10B, by using the storage unit 32 provided in the administrative server 30 or an external device connected via a network.

The request determination unit 34 compares instillation permission request information transmitted from the first electronic device 10A and the instillation permission information transmitted in advance from the second electronic device 10B and registered in the administrative server 30, and determines whether to transmit the instillation permission information to the first electronic device 10A.

When the request determination unit 34 determines that the instillation permission information is to be transmitted to the first electronic device 10A, the administrative server 30 transmits the instillation permission information to the first electronic device 10A.

### [3-3. Processing in Diagnosis Support Device 200 and Processing in Administrative Server 30]

Processing in the diagnosis support device 200 is similar to that of the second embodiment.

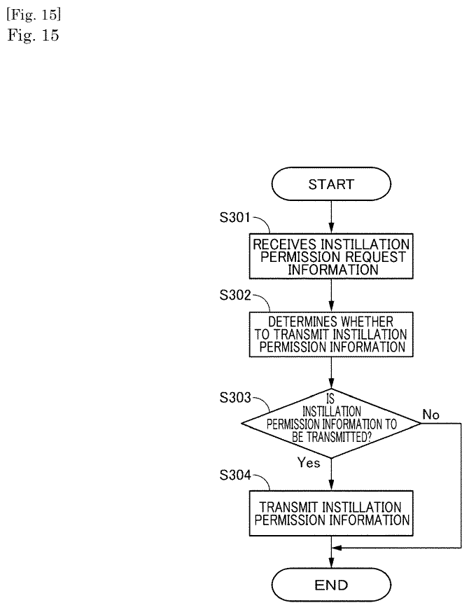

Referring to FIG. 15, processing in the administrative server 30 will be described below. It is assumed that the administrative server 30 preregisters the instillation permission information transmitted from the second electronic device 10B and stores the information in the storage unit 32.

First, in step S301, the administrative server 30 receives the instillation permission request information transmitted from the first electronic device 10A. Subsequently, in step S302, the request determination unit 34 determines whether to transmit the instillation permission information to the first electronic device 10A, on the basis of whether the instillation permission request information and the instillation permission information preregistered in the administrative server 30 agree with each other.

As described above, the instillation permission information includes a user ID, a patient ID, an ophthalmologist ID, eye left/right information, and a validity period, and the instillation permission request information includes a user ID, a patient ID, an ophthalmologist ID, eye left/right information, a mydriatic state determination result, a diagnosis result, diagnosis reliability, and an eye image. The request determination unit 34 determines whether all information items included in the instillation permission information agree with information items included in the instillation permission request information. When all information items included in the instillation permission information agree with information items included in the instillation permission request information, it is determined that the instillation permission information is to be transmitted to the first electronic device 10A.

The validity period is set by a validity start date and a validity end date. For the validity period, the request determination unit 34 determines whether the transmission date and time of an instillation permission request is set in a period from the validity start date to the validity end date of the validity period. If the validity period has only one day, the validity start date and the validity end date are set as the same date, or the validity period is set with a time.

When the request determination unit 34 determines that the instillation permission information is to be transmitted to the first electronic device 10A, the processing advances from step S303 to step S304 (Yes at step S303).

In step S304, the administrative server 30 transmits the instillation permission information to the first electronic device 10A.

When the request determination unit 34 determines that the instillation permission information is not to be transmitted to the first electronic device 10A, the processing is terminated (No at step S303).

The processing in the third embodiment is performed thus. The third embodiment can obtain the same effect as the second embodiment.

Furthermore, in the third embodiment, the ophthalmologist does not need to confirm the instillation permission request information in real time and determine whether to transmit the instillation permission information, thereby further reducing time constraints. Moreover, when the non-ophthalmologist photographs a patient's eye and the diagnosis support device 200 performs processing such as diagnosis of an eye disease, the ophthalmologist may perform another task and the second electronic device 10B does not need to be connected to the administrative server 30.

### 4. Modification Example

The embodiments of the present technique have been specifically described. The present technique is not limited to the foregoing embodiments and various modifications can be made on the basis of the technical idea of the present technique.

In the description of the embodiments, a target of diagnosis is an eye, a symptom to be diagnosed is cataract, and diagnostic treatment is the instillation of the mydriatic agent. The present technique is not limited thereto. For example, the target of diagnosis may be an eye, a symptom to be diagnosed may be diabetes in diagnosis based on a fundus image, or the diagnostic treatment may be administration of a contrast medium.

The target of diagnosis is not limited to an eye and may be another part of a human body. For example, the target of diagnosis may be a stomach, the state of the target of diagnosis may be the visibility of the stomach, and the diagnostic treatment may be the spraying of coloring matters in real-time diagnosis using a gastro camera. The spraying of coloring matters is a method for spraying blue liquid (e.g., indigocarmine) over a stomach to facilitate diagnosis.

The present technique can be also configured as follows:
(1)

A diagnosis support device including:
a state determination unit that determines a state of a target of diagnosis; and
a treatment necessity determination unit that determines whether diagnostic treatment is necessary on the basis of the state of the target of diagnosis.
(2)

The diagnosis support device according to (1), wherein the treatment necessity determination unit determines whether the diagnostic treatment is necessary on the basis of the result of diagnosis on the target of diagnosis.
(3)

The diagnosis support device according to (2), wherein the treatment necessity determination unit determines whether the diagnostic treatment is necessary on the basis of the reliability of the diagnosis result.
(4)

The diagnosis support device according to any one of (1) to (3), further including an output processing unit that outputs an instruction on the basis of the determination result of the treatment necessity determination unit.
(5)

The diagnosis support device according to (4), wherein when the treatment necessity determination unit determines that the diagnostic treatment is necessary, the output processing unit outputs an instruction to perform the diagnostic treatment.
(6)

The diagnosis support device according to (4), wherein when the treatment necessity determination unit determines that the diagnostic treatment is not necessary, whether an examination by a doctor is necessary is determined on the basis of the stage of a disease indicated by the diagnosis result.
(7)

The diagnosis support device according to (6), wherein when the treatment necessity determination unit determines that an examination by the doctor is necessary, the output processing unit outputs a consultation instruction.
(8)

The diagnosis support device according to any one of (1) to (7), further including a diagnosis processing unit that diagnoses the target of diagnosis.
(9)

The diagnosis support device according to (8), wherein the diagnosis processing unit conducts a diagnosis using a plurality of machine learning models and switches the diagnostic models on the basis of a state of the target of diagnosis.
(10)

The diagnosis support device according to any one of (1) to (9), further including a diagnosis target detection unit that detects the target of diagnosis from an image.
(11)

The diagnosis support device according to any one of (1) to (10), wherein the target of diagnosis is an eye.
(12)

The diagnosis support device according to (11), wherein the state of the target of diagnosis is whether the eye is placed in a mydriatic state.
(13)

The diagnosis support device according to any one of (1) to (12), wherein the diagnostic treatment is the instillation of a mydriatic agent.
(14)

The diagnosis support device according to any one of (1) to (13), further including an instillation permission request transmission unit that transmits a request for obtaining permission to perform the diagnostic treatment, when the treatment necessity determination unit determines that the diagnostic treatment is
(15)

The diagnosis support device according to (14), wherein the permission to perform the diagnostic treatment is received in response to the request, the permission being transmitted from a device used by a doctor.
(16)

The diagnosis support device according to (13), wherein the permission to perform the diagnostic treatment is received in response to the request, the permission being transmitted from a server device.
(17)

The diagnosis support device according to (16), wherein the permission to perform the diagnostic treatment is transmitted to the server device from a device used by an ophthalmologist and is preregistered in the server device.
(18)

A diagnosis support method including: determining a state of a target of diagnosis; and
determining whether diagnostic treatment is necessary on the basis of the state of the target of diagnosis.
(19)

A program that causes a computer to perform a diagnosis support method including: determining a state of a target of diagnosis; and
determining whether diagnostic treatment is necessary on the basis of the state of the target of diagnosis.

### REFERENCE SIGNS LIST

101 Eye detection/left-right eye determination unit
103 State determination unit
104 Diagnosis processing unit
105 Treatment necessity determination unit
106 Output processing unit
100, 200 Diagnosis support device
201 Instillation permission request transmission unit
30 Administrative server

1. A diagnosis support device comprising: a state determination unit that determines a state of a target of diagnosis; and
a treatment necessity determination unit that determines whether diagnostic treatment is necessary on a basis of the state of the target of diagnosis.

2. The diagnosis support device according to claim 1, wherein the treatment necessity determination unit determines whether the diagnostic treatment is necessary on a basis of a result of diagnosis on the target of diagnosis.

3. The diagnosis support device according to claim 2, wherein the treatment necessity determination unit determines whether the diagnostic treatment is necessary on a basis of reliability of the diagnosis result.

4. The diagnosis support device according to claim 1, further comprising an output processing unit that outputs an instruction on a basis of a determination result of the treatment necessity determination unit.

5. The diagnosis support device according to claim 4, wherein when the treatment necessity determination unit determines that the diagnostic treatment is necessary, the output processing unit outputs an instruction to perform the diagnostic treatment.

6. The diagnosis support device according to claim 4, wherein when the treatment necessity determination unit determines that the diagnostic treatment is not necessary, whether an examination by a doctor is necessary is determined on a basis of a stage of a disease indicated by the diagnosis result.

7. The diagnosis support device according to claim 6, wherein when the treatment necessity determination unit determines that an examination by the doctor is necessary, the output processing unit outputs a consultation instruction.

8. The diagnosis support device according to claim 1, further comprising a diagnosis processing unit that diagnoses the target of diagnosis.

9. The diagnosis support device according to claim 8, wherein the diagnosis processing unit conducts a diagnosis using a plurality of machine learning models and switches the diagnostic models on a basis of a state of the target of diagnosis.

10. The diagnosis support device according to claim 1, further comprising a diagnosis target detection unit that detects the target of diagnosis from an image.

11. The diagnosis support device according to claim 1, wherein the target of diagnosis is an eye.

12. The diagnosis support device according to claim 11, wherein the state of the target of diagnosis is whether the eye is placed in a mydriatic state.

13. The diagnosis support device according to claim 1, wherein the diagnostic treatment is instillation of a mydriatic agent.

14. The diagnosis support device according to claim 1, further comprising an instillation permission request transmission unit that transmits a request for obtaining permission to perform the diagnostic treatment, when the treatment necessity determination unit determines that the diagnostic treatment is necessary.

15. The diagnosis support device according to claim 14, wherein the permission to perform the diagnostic treatment is received in response to the request, the permission being transmitted from a device used by a doctor.

16. The diagnosis support device according to claim 13, wherein the permission to perform the diagnostic treatment is received in response to the request, the permission being transmitted from a server device.

17. The diagnosis support device according to claim 16, wherein the permission to perform the diagnostic treatment is transmitted to the server device from a device used by an ophthalmologist and is preregistered in the server device.

18. A diagnosis support method comprising: determining a state of a target of diagnosis; and
determining whether diagnostic treatment is necessary on a basis of the state of the target of diagnosis.

19. A program that causes a computer to perform a diagnosis support method comprising: determining a state of a target of diagnosis; and
determining whether diagnostic treatment is necessary on a basis of the state of the target of diagnosis.
